(19) United States
(12) Patent Application Publication
(10) Pub. No.: US 2023/0338206 A1
(43) Pub. Date: Oct. 26, 2023

(54) INCONTINENCE AND MENSTRUAL APPAREL

(71) Applicant: The Period Company, Los Angeles, CA (US)

(72) Inventors: Karla Welch, Los Angeles, CA (US); Jennifer Terry, Los Angeles, CA (US)

(21) Appl. No.: 18/026,584

(22) PCT Filed: Sep. 9, 2021
(86) PCT No.: PCT/US21/49739

§ 371 Date: Mar. 15, 2023

Related U.S. Application Data
(60) Provisional application No. 63/079,973, filed on Sep. 17, 2020.

(51) Int. Cl. A61F 13/496; A61F 13/511; A61L 15/40; A41D 31/102; A41B 9/00; A41D 1/00

(52) U.S. Cl. CPC A61F 13/496; A61F 13/51121; A61L 15/40; A41D 31/102; A41B 9/001; A41D 1/00; A61F 2013/530299

(57) ABSTRACT

Disclosed are apparel designed for use during menstrual cycle, incontinence or when involuntary discharge of bodily fluids is expected. Various styles of absorbent undergarments with various layering and structures therein are disclosed. In one embodiment, the disclosed absorbent undergarments include one or more absorbent liners. The absorbent liners can have one or more hydrophilic layers and one or more breathable, waterproof layers in the crotch region of the undergarments.

### CROSS-REFERENCE TO RELATED APPLICATIONS

This application claims the benefit of priority of U.S. Provisional Application No. 63/079,973 filed on Sep. 17, 2020, entitled “INCONTINENCE AND MENSTRUAL APPAREL,” content of which is incorporated herein by reference in its entirety and should be considered a part of this specification.

### BACKGROUND

### Field

This invention relates generally to the field of apparel, and more particularly to undergarments designed to absorb and contain involuntary discharge of fluids to protect outer clothing of a wearer of the undergarments.

### Description of the Related Art

Various styles of absorbent undergarments exist to assist persons who experience involuntary discharge of bodily fluids, such as urine or menses. Pads, tampons, and other disposable products are also used. Some existing products combine the use of disposable materials and washable undergarments. With increased awareness and desire for reducing human waste, interest in reusable menstrual and incontinence undergarment has increased. Some existing washable absorbent undergarments use thick paddings or highly absorbent inserts in the crotch region, which can make them bulky and uncomfortable to wear. Others are thinner and more comfortable to wear but may not be sufficiently absorbent, such that their users may have to supplement them with traditional disposable products, such as pads or tampons. Consequently, there is a need for improved absorbent undergarments that utilize material and structures that are comfortable and natural to wear, while providing improved absorption capacity.

### SUMMARY

In one aspect an undergarment is disclosed. The undergarment can include: a waist opening; first and second leg openings; a main body portion made of a main body fabric, the main body portion comprising a crotch portion, wherein the crotch portion comprises portions of the first and second leg openings; the crotch portion further comprising an absorbent liner, the absorbent liner comprising: a top layer; one or more intermediate hydrophilic layers; a breathable and waterproof layer; a self-fabric layer made of the main body fabric.

In one aspect another absorbent undergarment is disclosed. The absorbent undergarment can include: a waist opening; first and second leg openings; a main body fabric extending to all regions of the absorbent undergarment and comprising a bottom layer of the absorbent undergarment, wherein the bottom layer comprises the outside surfaces of the absorbent undergarment; and an absorbent liner in a crotch region of the absorbent undergarment, the absorbent liner comprising: a top layer; one or more intermediate hydrophilic layers; a breathable, waterproof layer; and the bottom layer, wherein the top and bottom layers envelope the one or more hydrophilic layers and the breathable, waterproof layer.

### BRIEF DESCRIPTION OF THE DRAWINGS

These drawings and the associated description herein are provided to illustrate specific embodiments of the invention and are not intended to be limiting.

FIGS. 1A-1D illustrate an absorbent undergarment according to an embodiment, in a garment style termed, hipster.

FIGS. 2A-2C illustrate an absorbent undergarment according to an embodiment, in a garment style termed, brief.

FIG. 3 illustrates an absorbent undergarment according to an embodiment, in a garment style termed, sleeper boyshort.

FIGS. 4A-4D illustrate an absorbent undergarment according to an embodiment, in a garment style termed, boxer.

FIGS. 5A-5D illustrate an absorbent undergarment according to an embodiment, in a garment style termed, bikini.

FIGS. 6A-6D illustrate an absorbent undergarment according to an embodiment, in a garment style termed, boyshort.

FIGS. 7A-7D shows another absorbent undergarment, in the style of boxers, according to an embodiment.

FIGS. 8A-8B illustrate an absorbent undergarment, according to an embodiment, termed adaptive bikini.

FIGS. 9A-9B illustrates an absorbent undergarment according to an embodiment, in a garment style termed, thong.

FIGS. 10A-10B illustrates an alternative embodiment of the sleeper boyshort style absorbent undergarment shown in FIG. 3.

FIGS. 11A-11B illustrates an alternative embodiment of the boxer style absorbent undergarment, shown in FIGS. 4A-4D.

### DETAILED DESCRIPTION

The following detailed description of certain embodiments presents various descriptions of specific embodiments of the invention. However, the invention can be embodied in a multitude of different ways as defined and covered by the claims. In this description, reference is made to the drawings where like reference numerals may indicate identical or functionally similar elements.

Unless defined otherwise, all terms used herein have the same meaning as are commonly understood by one of skill in the art to which this invention belongs. All patents, patent applications and publications referred to throughout the disclosure herein are incorporated by reference in their entirety. In the event that there is a plurality of definitions for a term herein, those in this section prevail. When the terms “one”, “a” or “an” are used in the disclosure, they mean “at least one” or “one or more”, unless otherwise indicated.

A variety of absorbent undergarments exist that are intended for use during menstruation cycle or by persons suffering from incontinence. Existing absorbent undergarments offer a variety of solutions. Quick dry materials and fabrics such as polyester or nylon are used in some existing products. Others use a disposable or permanent pad inserted in a pocket or chamber in the crotch region of an absorbent undergarment. While quick-dry material can be useful in keeping the skin and body of the wearer dry, the synthetic material can be uncomfortable for long-term wear. For example, such products can be useful in active or sportswear, but may be uncomfortable for an undergarment worn for long hours. At the same time, some existing products that use an internal pad or padded section can be bulky and can feel unnatural because they fail to adequately conform to the shape of the wearer's body compared to normal undergarments. Some wearers can be hesitant to use thick or overly padded absorbent undergarments, because they may feel they are wearing adult size diapers. Similarly, persons identifying with a masculine gender identity, can be hesitant to use underwear or pads that can also be used by females or can be deemed as feminine-related products.

Furthermore, huge amounts of wastes are generated by incontinence or menstrual products that use disposable material. Many wearers of absorbent undergarments are naturally interested in preserving the environment and reducing their carbon footprint by wearing reusable and launderable absorbent products, instead of disposable ones to reduce human waste.

Existing products that use padding can provide high absorbency but can also be too thick for comfortable and natural wear for long hours. At the same time, many existing absorbent undergarments are too thin and have limited absorbent capacity to be useful as a stand-alone menstrual or incontinence undergarment and may have to be supplemented by disposable pads or other similar and more absorbent products. Furthermore, existing products that use a synthetic layer adjacent to the skin can also feel unnatural or uncomfortable, especially if worn for long hours. At the same time, persons experiencing involuntary fluid discharge can be in any physical position when the discharge occurs. For example, the person can be lying down, sitting or in motion. A desirable absorbent undergarment can catch and contain the discharge from reaching the outer clothing regardless of the wearer's body position.

In part, to address comfort issues with existing designs, some products use thinner fabric layers and fewer fabric layers coupled with a method of creating seams that includes heat bonding of an elastic at the edges of the different fabric layers. However, these products can suffer from substantially reduced absorption capacity to be useful as stand-alone absorbent undergarment. Many wearers of such products would still need to continue using pads or inserts as back-up. Furthermore, some of the existing products allow the bodily fluids to run through the layers of the undergarment (and even run along waterproof surfaces), reach the fabric seams and exit through the seams, soiling the wearer's outer garments. Also, many existing absorbent undergarment designs, due to thickness or padding, cannot be incorporated in a stylish and normal-looking undergarment that would encourage their adoption and use. Consequently, there is a need for improved absorbent undergarments that can address the issues outlined above.

The described embodiments strike a balance between undergarment comfort and absorption capacity and can be incorporated in stylish designs. They include use of a layered fabric structure to provide fluid absorption and retention, as opposed to using pockets or chambers in the crotch region, which can often lead to unnatural and uncomfortable fit. The described undergarments can include different sections and panels with different fabric layering and different materials in the different sections, in order to provide for stylish, yet absorbent undergarments that can catch and retain fluid discharge, regardless of the wearer's body position. In some embodiments, one or more absorbent liners are provided that are part of the crotch region of an absorbent undergarment or can be included in other parts of the undergarment.

### EMBODIMENTS

Various styles and designs of undergarment can incorporate the described embodiments. Persons of ordinary skill in the art can appreciate that the described embodiments are not so limited and can be incorporated in other undergarment designs and even other garments, not limited to undergarments. Furthermore, the described embodiments can be incorporated in styles appealing to any gender and segment of the population, for example, men, women, transgenders, non-binary menstruating persons, and anyone suffering from incontinence. Furthermore, the described embodiments can be used in any situation, where it is desired to catch, retain and prevent leakage of involuntary bodily fluids in a garment or undergarment. For example, the embodiments may be used in period underwear, baby diapers, or incontinence underwear. Furthermore, the described embodiments can be used in garments of various material, including for example, natural, synthetic or blended material. The embodiments will now be described in reference to the accompanied drawings and the exemplary styles described below.

Hipster

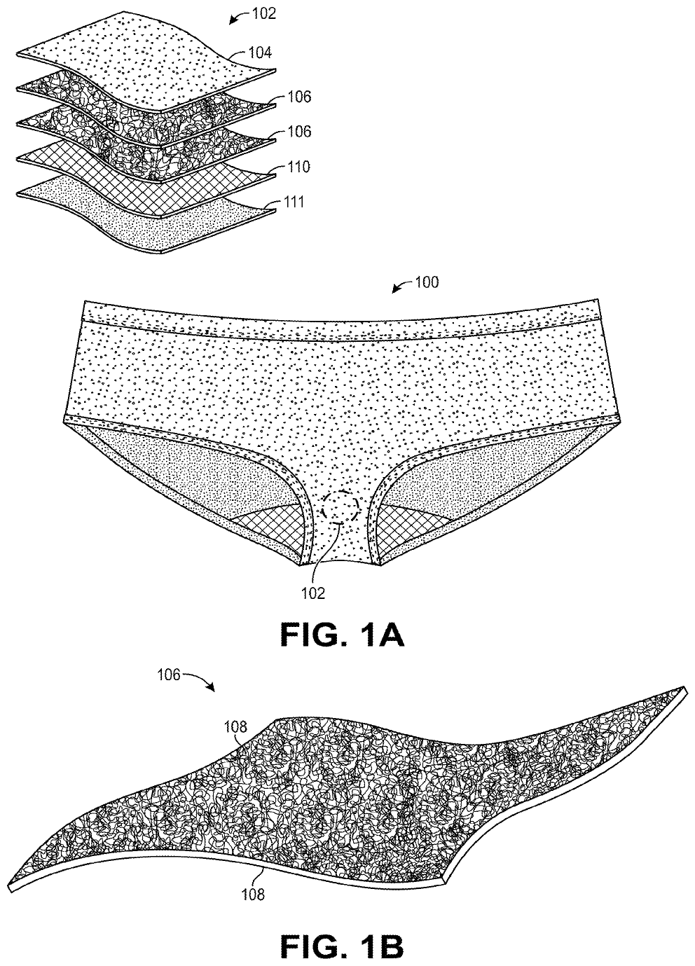

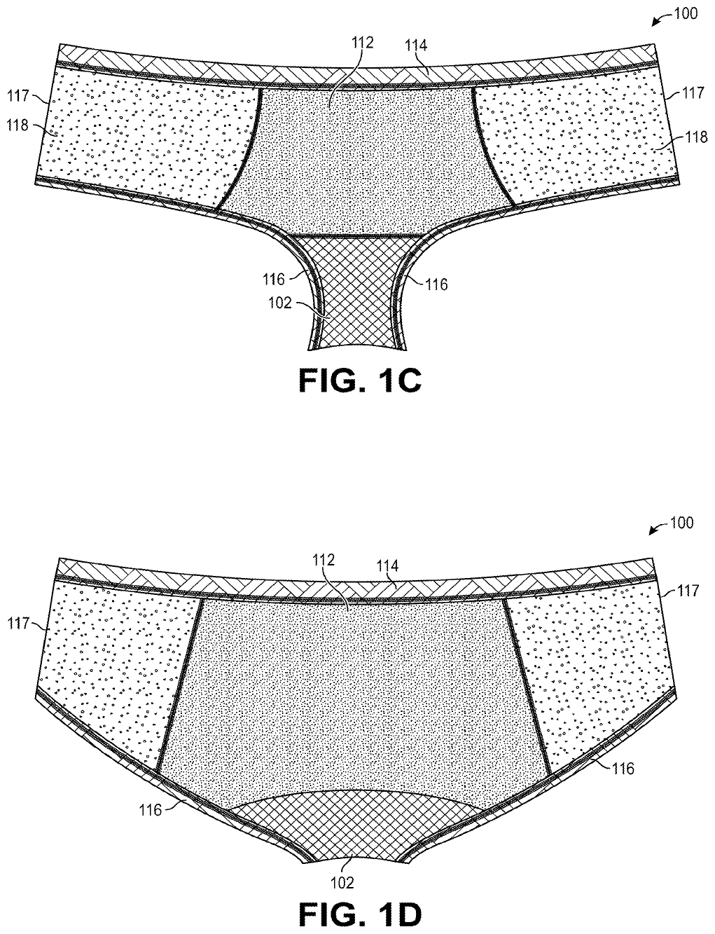

FIGS. 1A-1D illustrate an absorbent undergarment 100 according to an embodiment, in a garment style termed, hipster, which can be used by women, but other gender groups can also use this undergarment style. FIG. 1A illustrates an exemplary style in which the absorbent undergarment 100 can be implemented. The absorbent undergarment 100 can have an absorbent liner 102 in the crotch region. The absorbent liner 102 can include multiple layers of fabric in the crotch region to catch, retain and/or contain discharged fluids. The absorbent liner 102 can include a top layer 104, which can be the closest fabric layer to the skin of the wearer in the interior surface of the absorbent undergarment 100, relative to other fabric layers. The top layer 104 can be made of various fabrics, natural, synthetic or a blend of natural and synthetic. For example, the top layer can be a cotton and spandex blend. A high percentage cotton can allow the top layer closest to the skin to be comfortable and absorbent at the same time, helping to transfer the moister to layers below it. In a preferred embodiment of the absorbent undergarment 100, the top layer 104 is 95% cotton, 5% spandex. High percentage cotton top layer can be more performant in menstrual products, as cotton interacts better with blood. Conversely, synthetic top layers (e.g., polyester) are more performant in products that are meant to absorb urine, such as in incontinence undergarments. A high percentage of a material in a blended fabric can refer to the fabric having at least 80% of the high percentage material in the blend. For example, a high percentage cotton and spandex blend can refer to the fabric having at least 80% cotton in the cotton and spandex blend.

The absorbent liner 102 can further include one or more intermediate hydrophilic layers (IHL) 106. The IHL 106 can be made of synthetic absorbent material that can absorb and transfer moisture and fluids from the top layer side to the more water-resistant and/or waterproof layers underneath. In one embodiment, the IHL 106 can be made of a towel-like fabric, which has a textured surface area, resulting in more surface area than the top layer. Fluids tend to move from areas of high concentration to areas of low concentration. Consequently, increased surface area in the IHL 106 can result in better moisture absorption and transfer in those layers as fluids have more surface area to occupy. In one embodiment, the textured surface area of the IHL 106 can exist on both sides of the IHL 106, FIG. 1B illustrates an example IHL 106. The IHL 106 can include a textured surface 108 on one or both sides of the IHL 106. The textured surface 108 can be like the surface of a towel fabric and may be made of looped strands of fabric. Woven or knitted textures can also be used. In one embodiment, the IHL 106 can be a double-sided velour fabric (e.g., as opposed to French Terry style of fabric, which is only textured on one side), where textured surface 108 exists on both sides of the IHL 106. Various synthetic or natural fabric materials may be used for the IHL 106. In a preferred embodiment, the IHL 106 is a polyester fabric. In some embodiments, the IHL 106 has a weight of approximately 200 grams per square meter (GSM).

The IHL 106 is sandwiched between the top layer and a breathable, water-resistant, or waterproof layer (BWL) 110. The top layer 104 and the IHL 106 absorb and transfer moisture to the breathable, waterproof layer 110. In a preferred embodiment, the BWL 110 is made of a knit polyester fabric that is laminated with a polyurethane film on the side that is opposite the IHL 106 side. In other words, the BWL 110 includes an absorbent side that is touching, adjacent or abutting the IHL 106 and further includes a polyurethane laminate side that is opposite the IHL 106 side. In some embodiments, the BWL can be a polyurethane laminate (PUL) layer, having a weight of approximately 150 GSM.

Below the BWL 110, the absorbent liner 102 includes the bottom layer 111. The bottom layer 111 can be made of self-fabric material. The self-fabric can refer to the main fabric of the absorbent undergarment 100. In other words, the self-fabric is the majority fabric of the absorbent undergarment 100 and can be made of a variety of material, depending on the usage and style of the absorbent undergarment 100. Examples include, natural, synthetic or blend material, such as cotton, spandex, polyester, microfiber, hemp, bamboo and others. In some embodiments, the bottom layer 111 can be the same fabric material as the top layer 104. For example, in absorbent undergarment 100, the bottom layer can be a cotton and spandex blend with a high cotton percentage (e.g., 95% cotton, 5% spandex). Furthermore, where cotton is disclosed in the description herein, organic cotton can also be used in addition to or instead of cotton.

The disclosed undergarments can be constructed having a plurality of regions, some regions of the disclosed undergarments can be multilayered (e.g., the absorbent liner 102), and other regions as will be disclosed herein may be made of fewer fabric layers or a single fabric layer. The disclosed multi-region constructions improve the functionality of the described undergarments and allow for cost-effective and attainable products. At the same time, the disclosed constructions are not cost-prohibitively complex, as to make the resulting products unattainable for a large portion of the population. By contrast, several existing absorbent undergarment products can be too costly or complex in their construction to be attainable or practical for a large portion of the population.

FIGS. 1C and 1D illustrate the structure and construction of the absorbent undergarment 100. The absorbent undergarment 100 can include front and back panels, relative to the body orientation of a wearer of the absorbent undergarment 100. FIG. 1C illustrates the interior front panel of the absorbent undergarment 100 and FIG. 1D illustrates the interior back panel of the absorbent undergarment 100. In some embodiments, the front and back panels generally resemble an hour-glass shape before they are sewn together to form the waist opening 114 and the leg openings 116 of the absorbent undergarment 100. Referring to FIG. 1C, the absorbent undergarment 100 can include a waist opening 114 and first and second leg openings 116. The absorbent liner 102 can be incorporated in the crotch region of the absorbent undergarment 100, where the absorbent liner 102 covers a portion of the crotch region of the absorbent undergarment 100, including a portion of the first and second leg openings 116, but not reaching all the way to the waist opening 114.

Furthermore, in the shown example, the main body fabric of the absorbent undergarment 100 can include a lined portion 112, where the main body fabric is lined with another layer of the main body fabric. The lined portion 112 is in a portion of the absorbent undergarment 100 above the crotch region, between the waist and leg openings 114, 116, wherein it extends to some parts of the waist and leg openings 114, 116, but it does not extend to all edges of the waist and leg openings 114, 116. In other words, the main body portion of the absorbent undergarment 100 in a portion extending between the waist opening 114, the leg openings 116 and the crotch portion is lined with an additional layer of the main body fabric, wherein the lined portions exist in both front and back panels only partially and not extending to all edges of the waist and leg openings 114, 116. Consequently, a single layered region 118 extends from the lined portion 112 to the waist and leg openings 114, 116. The single layered region 118 is made of a single layered fabric from the main body fabric.

Similarly, the interior back panel of the absorbent undergarment 100, shown in FIG. 1D includes the crotch region having the absorbent liner 102 and the lined portion 112 that extends from the absorbent liner 102 to some portions of the waist and leg openings 114, 116, but not all the way to the side edges 117 or to all edges of the waist and leg openings 114, 116 of the absorbent undergarment 100. Consequently, the interior back panel also includes the single layered region 118, extending from the side edges 117 to the lined portion 112 and covering portions of the waist and leg openings 116, 118 that are not covered by the lined portion 112 or the crotch region. In some embodiments, the lined portion 112 exists, relative to the waist opening 114, in approximately ⅓ of the perimeter of the waist opening 114 in the interior front panel and in approximately ½ the perimeter of the waist opening 114 in the interior back panel of the absorbent undergarment 100.

In one embodiment, the main body fabric is the bottom layer 110, that extends to all regions of the absorbent undergarment 100, including the single layered 118, the lined portion 112, and the crotch region. In some embodiment, the top layer 104 can also be a layer made of the main body fabric. In one embodiment, an elastic, for example, brushed elastic dyed to match (DTM) can be used in the waist opening 114 to sew along with other layers present in the waist opening 114. In one embodiment, a brushed elastic, DTM and ½ an inch in width, can be used in the waist opening 114 to sew along with other layers present therein. In some embodiments, the leg openings 116 can be sewn together along with fabric layers present therein using a folder-over elastic. In one embodiment, a fold-over elastic DTM of width of ⅜ of an inch can be used. The use of fold-over elastic and sewing technique can prevent an issue with existing absorbent undergarments in which moisture and fluids run along the fabric and leak through the exposed edges of the seams. Using fold-over elastic at the seams can reduce or minimize that issue.

Brief

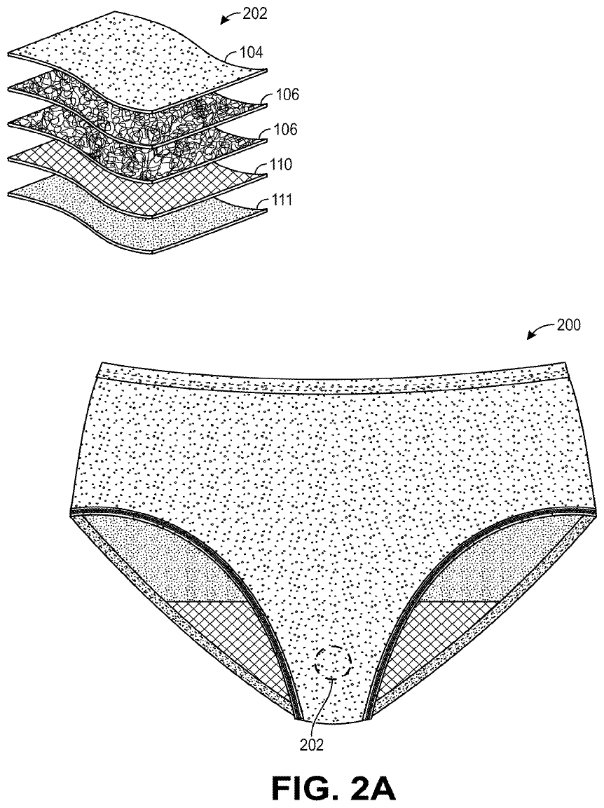

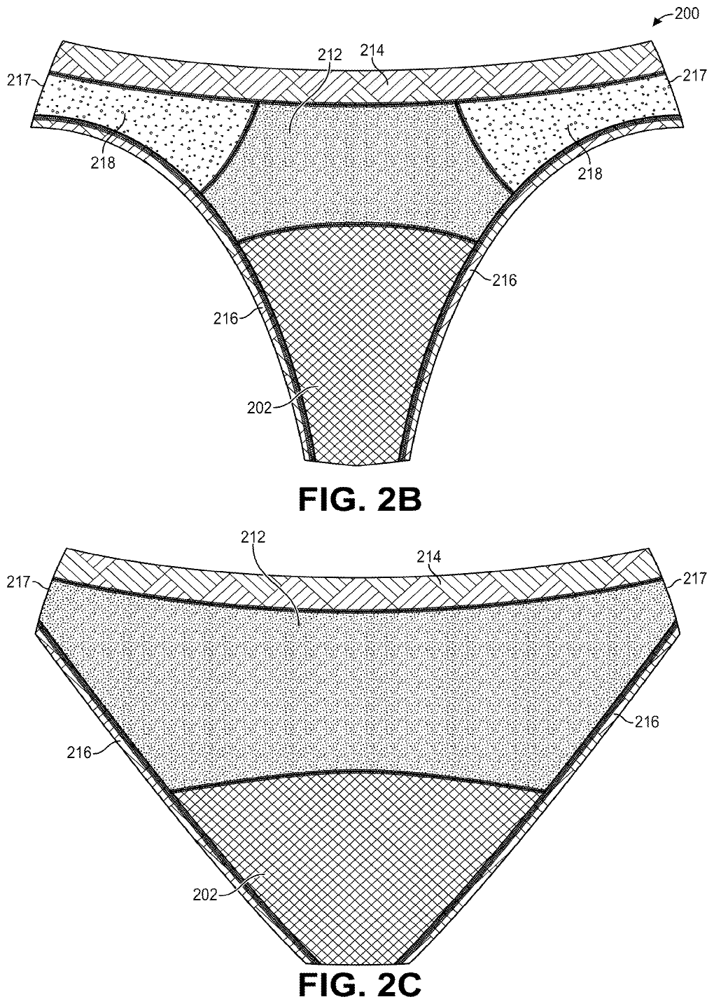

FIGS. 2A-2C illustrate an absorbent undergarment 200 according to an embodiment, in a garment style termed, brief, which can be used by women, but other gender groups can also use this undergarment style. FIG. 2A illustrates an exemplary style in which the absorbent undergarment 200 can be implemented. The absorbent undergarment 200 can have an absorbent liner 202 in the crotch region. The absorbent liner 202 can be similar in material and structure to the absorbent liner 102, as described above. The absorbent undergarment 200 includes an interior front panel, shown in FIG. 2B and an interior back panel, shown in FIG. 2C. The absorbent undergarment 200 can include a lined portion 212, which like the lined portion 112 can be constructed of an additional layer of the main body fabric from which the absorbent undergarment 200 is constructed. Like the absorbent undergarment 100, in the front panel, the absorbent undergarment 200 can include single layered regions 218 flanking the lined portion 212. In the front panel of the absorbent undergarment 200, shown in FIG. 2B, the lined portion 212 reaches some portions of the waist opening 214 and some portions of the leg openings 216. The remaining portions are covered by the single layered region 218.

In the interior back panel shown in FIG. 2C, the lined portion 212 extends to all edges of the waist opening 214 and to all edges of the leg openings 216, not reached or covered by the crotch region. In other words, in the back panel, the crotch region reaches and covers a portion of the leg openings 216 and the remaining edges of the leg openings 216 are covered by the lined portion 212. In some embodiments, the crotch region or the absorbent liner 202 includes ½ perimeter of the leg openings 216 and the other halves are covered by the lined portion 212. The lined portion 212 substantially covers the remaining surface area in the back panel, not covered by the crotch region and the absorbent liner 202. Elastics and fold-over elastics sewn in the seams can also be used in the waist and leg openings 214, 216. DTM technique and measurements, as disclosed above in relation to the absorbent undergarment 100 can also be used.

Sleeper Boyshort

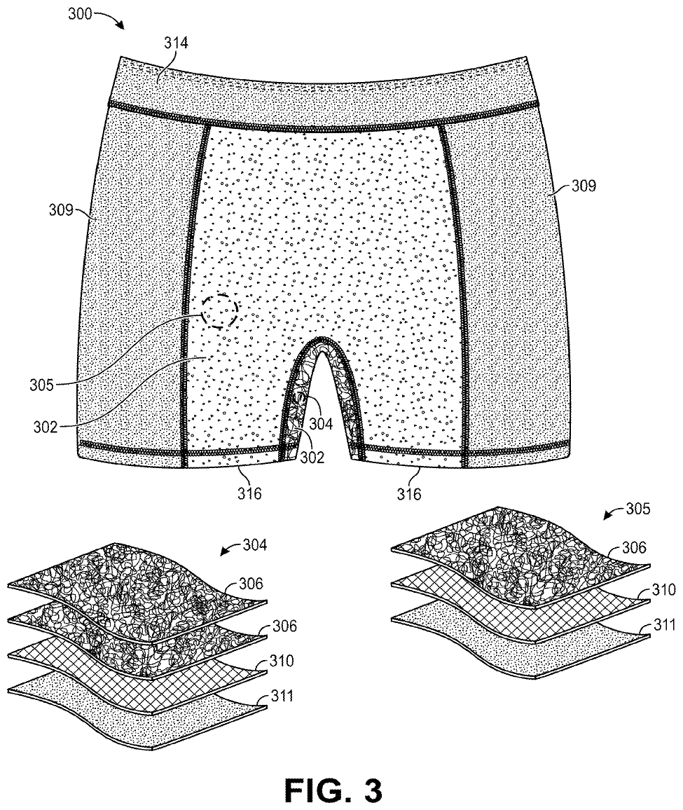

FIG. 3 illustrates an absorbent undergarment 300 according to an embodiment, in a garment style termed, sleeper boyshort, which can be used by women, but other gender groups can also use this undergarment style. The absorbent undergarment 300 can have an absorbent liner 302 in the crotch region. The absorbent liner 302 can include a gusset portion 304 and a lined portion 305. In one embodiment, the main fabric body of the absorbent undergarment 300 is made of a cotton and spandex blend, having a high percentage of cotton (e.g., 95% cotton and 5% spandex). The absorbent liner 302 can include top and bottom layers 309, 311 within which the gusset and lined portions 304, 305 are sandwiched. In some embodiments, both the top and bottom layers 309, 311 can be made of the self-fabric or the main body fabric (e.g., a 95% cotton, 5% spandex blend). Other high cotton percentages can also be used.

In some embodiments, the gusset portion 304 can include a plurality of layers such as two IHLs 306, which are similar in material and properties to the IHLs 106, described in relation the absorbent undergarment 100. The gusset portion 304 can also include a BWL 310, similar in material and properties to the BWL 110, described in relation to the embodiment of the absorbent undergarment 100. The gusset portion 304 can include the bottom layer made of self-fabric or the main body fabric of the absorbent undergarment 300, which in some embodiments can be a blend of cotton and spandex.

The absorbent liner 302 can also include a lined portion 305. The lined portion 305 can include a single IHL 306, a BWL 310 and a bottom layer, made of the self-fabric or main body fabric material of the absorbent undergarment 300. The lined portion 305 can extend from the gusset portion 304 to the waist and leg openings 314, 316 in partial edges of the waist and the leg openings 314, 316 and not extending to all edges of the waist and leg openings 314, 316. In some embodiments, the gusset portion 304 and the lined portion 305 extend to a perimeter of the waist opening and leg openings 314, 316, comprising approximately a ½ of the perimeter of the waist and leg openings 314, 316. The remaining areas of the absorbent undergarment 300 can be a single layered fabric.

Boxer

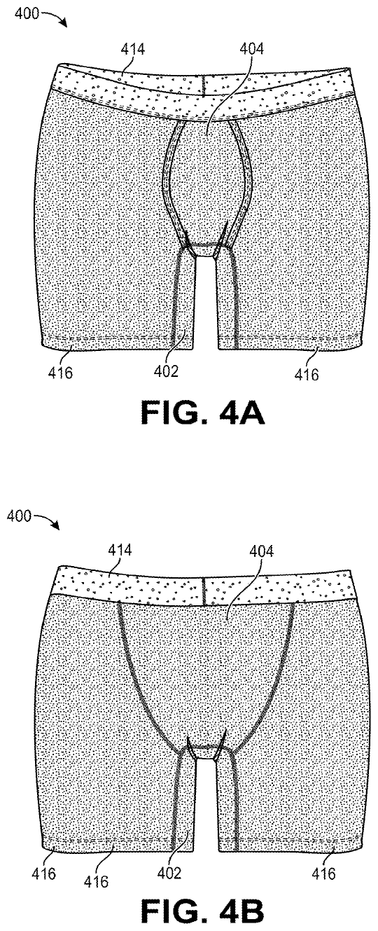

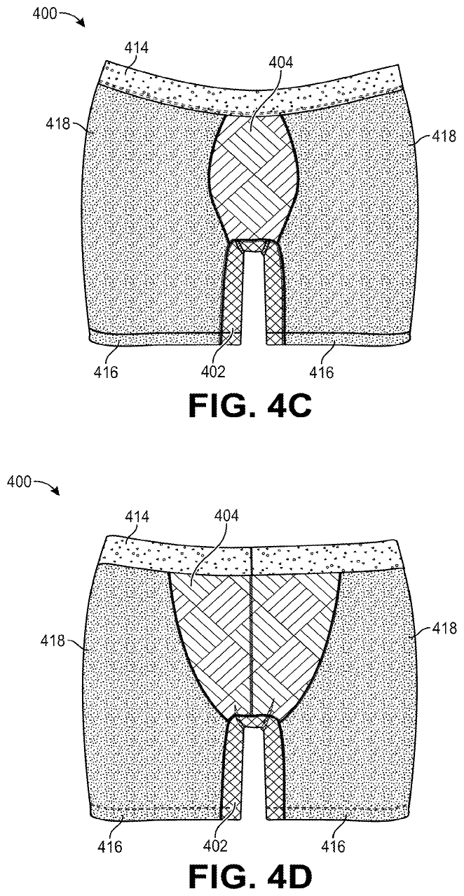

FIGS. 4A-4D illustrate an absorbent undergarment 400 according to an embodiment, in a garment style termed, boxer, which can be used by transgender women, but other gender groups can also use this undergarment style. FIG. 4A illustrates an exterior view of the front of the absorbent undergarment 400. FIG. 4B illustrates an exterior view of the back of the absorbent undergarment 400. Front and back in this context are used relative to the body direction of the wearer of the absorbent undergarment 400. The absorbent undergarment 400 can have a waist opening 414 and first and second leg openings 416. The absorbent undergarment 400 can have a first absorbent liner 402, as a gusset in the crotch region and a second absorbent liner 404, extending between the first absorbent liner 202 and the waist opening 414. The second absorbent liner 404 in the back of the absorbent undergarment 400 extends to a larger area than it does in the front of the absorbent undergarment 400. In both front and back panels of the absorbent undergarment 400, the second absorbent liner 404, only partially covers a region between the crotch region and the waist opening 414. In one embodiment, the waist opening 414 and the fabrics meeting therein can be sewn together along with a brushed elastic (e.g., having width of approximately 1.75 inches). In one embodiment, the leg openings 416 can be sewn together along with fabric layers that meet therein using a cover stitch (e.g., having width of approximately ⅝ of an inch).

FIGS. 4C and 4D illustrate interior views of the front and back panels of the absorbent undergarment 400, respectively. The first absorbent liner 402 can include a plurality of layers. In one embodiment, the layering, structure, materials, and properties of the first absorbent liner 402 is like the layering, structure and materials of the absorbent liner 202, as described above in relation to the embodiment of the absorbent undergarment 200. For example, the first absorbent liner 402 can include a top layer made of a cotton and spandex blend with high percentage of cotton (e.g., 95% cotton, 5% spandex), a first and second IHLs, a BWL, for example, a polyurethane laminate (PUL) layer, and a bottom layer comprising the self-fabric, which can also be a cotton, spandex blend of high percentage cotton.

The second absorbent liner 404 can include a plurality of layers, but may include fewer layers than the first absorbent liner 402 as moisture exposure in the area of the second absorbent liner 404 can be less than the moisture exposure in the area of the first absorbent liner 402. The absorbent liner 404 can include a top layer made of a cotton and spandex blend, having a high percentage of cotton (e.g., 95% cotton, 5% spandex), a single IHL, BWL and a bottom layer, comprising the self-fabric.

The materials and properties of the IHL and BWL can be chosen like the material and properties described above in relation to the absorbent undergarments 100, 200 and 300. In one embodiment, the top layer and the bottom layer are both made of self-fabric material (e.g., a high percentage cotton and spandex blend). In some embodiments, the bottom layer, self-fabric is the same layer and the same fabric as the main body fabric, upon which the other layers are sewn or otherwise assembled.

As illustrated in FIGS. 4C and 4D, the first absorbent liner 402 is a gusset in the crotch region of the absorbent undergarment 400. In the front panel of the absorbent undergarment 400, shown in FIG. 4C, the second absorbent liner 404 extends between the crotch region and the waist opening 414, in a shape roughly resembling an ellipse with waist and leg openings 414, 416 intersecting and terminating the elliptical shape. The remaining areas of the absorbent undergarment 400 not covered by the first and second absorbent liners 402, 404 are single layered regions 618 and can be made of the main body fabric.

In some embodiments, the single layered regions 418 and the bottom layers of the first and second absorbent liners 402, 404 are the same layer and the same fabric. Likewise, the first and second absorbent liners 402 and 404 can also share a single layer of IHL, a single layer of BWL and the same top layer, with the first absorbent liner 402 having an extra layer of IHL. In other words, the layers that are common between various regions of the disclosed absorbent undergarments can be cut to extend to and between their common regions and sewn or otherwise attached as such. If a region has additional layers, those layers can be then added on top or below the commonly shared layers and fabrics.

The second absorbent liner 404 in the back panel of the absorbent undergarment 400, as shown in FIG. 4D, also extends from the first absorbent liner 402 to the waist opening 414. However, the second absorbent liner 404 extends to a larger region in the back panel of the absorbent undergarment 400, compared to the area it extends to in the front panel of the absorbent undergarment 400. In one embodiment, the absorbent liner 404 in the back panel of the absorbent undergarment 400 extends to a region roughly resembling a shape of a triangle with the waist and leg openings 414, 416 intersecting and terminating the triangular shape.

Bikini

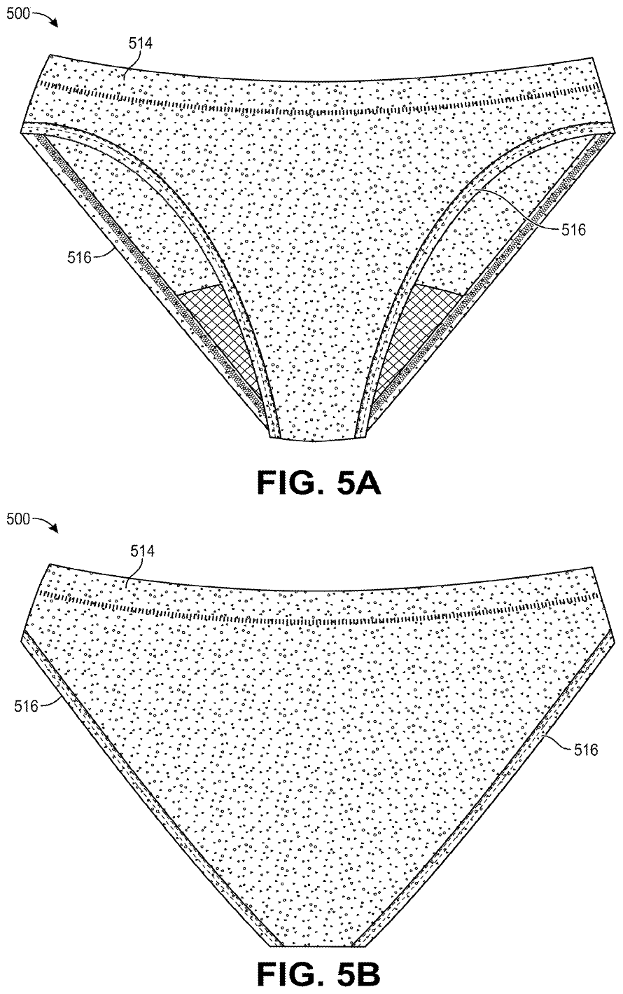

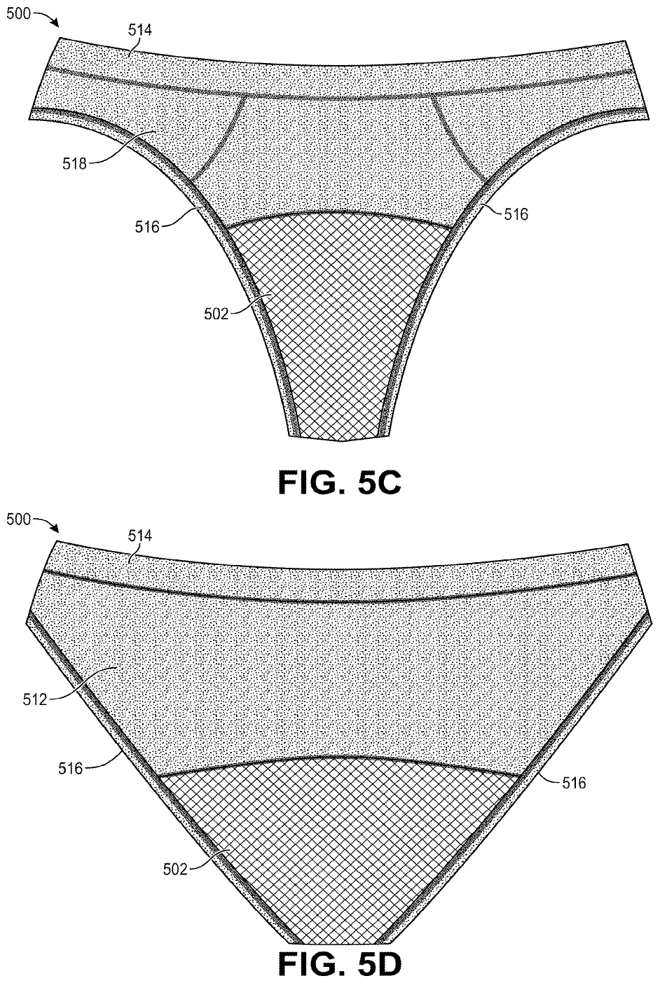

FIGS. 5A-5D illustrate an absorbent undergarment 500 according to an embodiment, in a garment style termed, bikini. FIG. 5A illustrates an exterior view of the front of the absorbent undergarment 500. FIG. 5B illustrates an exterior view of the back of the absorbent undergarment 500. Front and back in this context are used relative to the body direction of the wearer of the absorbent undergarment 500. The absorbent undergarment 500 can have a waist opening 514 and a first and second leg openings 516. In one embodiment, the waist opening 514 can be sewn together and along with fabric layers present therein using a folded waistband with blind hem stitching. In some embodiments, a ¾ of an inch folded waistband with blind hem stitching can be used. In some embodiments, the leg openings 516 and the fabric layers joined therein can be sewn using a “self-fabric binding” technique. For example, a ⅜ of an inch self-fabric binding at leg opening 516 can be used.

FIGS. 5C and 5D illustrate interior views of the front and back panels of the absorbent undergarment 500, respectively. The absorbent undergarment 500 can include an absorbent liner 502 in both front and back panels. The absorbent liner 502 can include a plurality of layers. In one embodiment, the layering, structure, materials, and properties of the absorbent liner 502 is like the layering, structure, and materials of the absorbent liner 202, as described above in relation to the embodiment of the absorbent undergarment 200. For example, the absorbent liner 502 can include a top layer made of a cotton and spandex blend with high percentage of cotton (e.g., 95% cotton, 5% spandex), a first and second IHLs, a BWL, for example, a PUL layer, and a bottom layer comprising the self-fabric, which can also be a cotton, spandex blend of high percentage cotton.

In the front panel, shown in FIG. 5C, the absorbent undergarment 500 can include a single layer region 518 extending from the absorbent liner 502 to the waist opening 514. In some embodiments, the fabric making up the single layer region 518 is the main body fabric from which the absorbent undergarment 500 is constructed and comprises the bottom layer or the self-fabric layer of the absorbent liner 502. Additional layers of the absorbent liner 502 can be laid upon, assembled, or otherwise constructed upon the bottom layer.

In the interior back panel of the absorbent undergarment 500, shown in FIG. 5D, the absorbent undergarment 500 can include a lined portion 512, which can be constructed of an additional layer of the main body fabric on top of the main body fabric. In other words, in the lined portion 512, the main body fabric or self-fabric is double layered. The lined portion 512 extends to all edges of the waist opening 514 and to all edges of the leg openings 516, not reached or covered by the crotch region. In other words, in the back panel, the crotch region reaches and covers a portion of the leg openings 516 and the remaining edges of the leg openings 516 are covered by the lined portion 512. In some embodiments, the crotch region, or the absorbent liner 502, includes approximately ½ perimeter of the leg openings 516 and the other halves are covered by the lined portion 512. In other words, the lined portion 512 substantially covers the remaining surface area in the back panel, not covered by the crotch region and the absorbent liner 502.

Boyshort

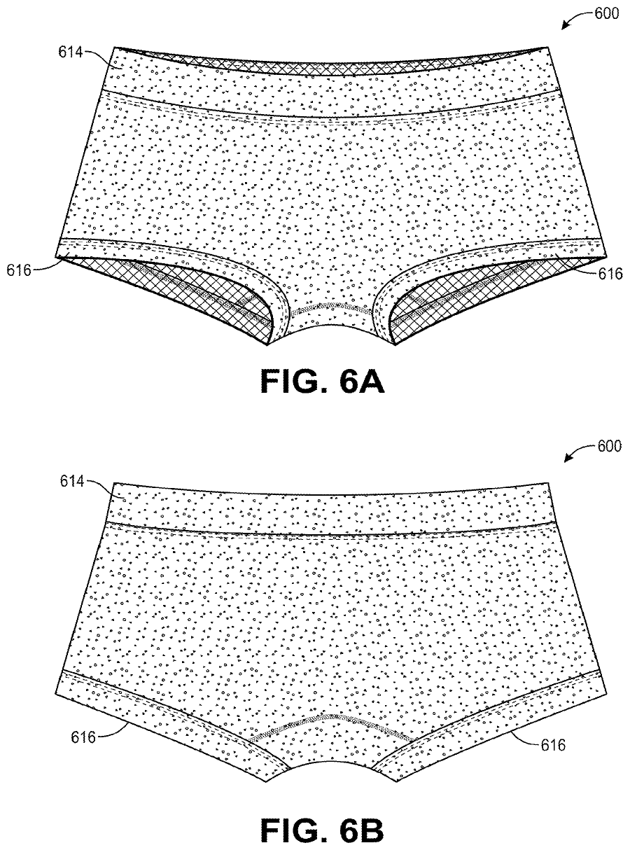

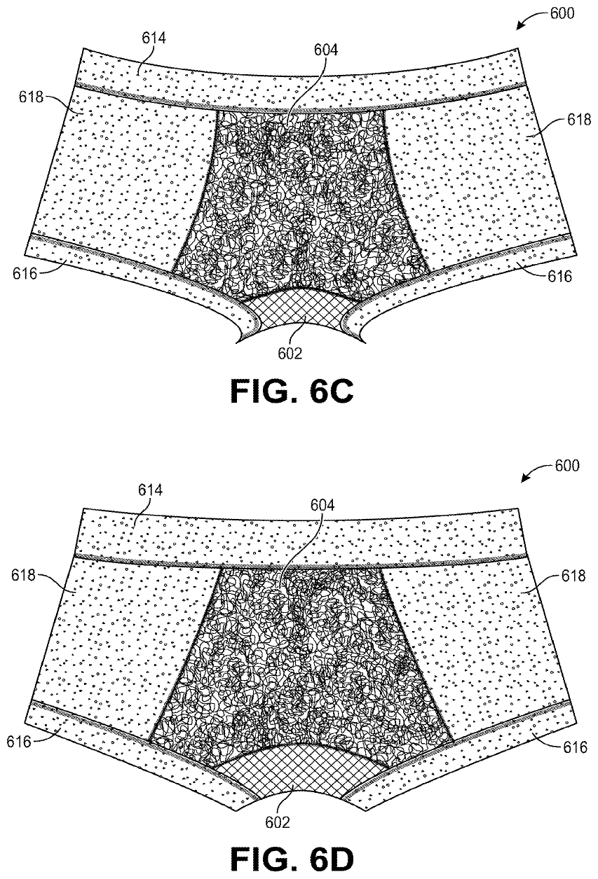

FIGS. 6A-6D illustrate an absorbent undergarment 600 according to an embodiment, in a garment style termed, boyshort. FIG. 6A illustrates an exterior view of the front of the absorbent undergarment 600. FIG. 6B illustrates an exterior view of the back of the absorbent undergarment 600. Front and back in this context are used relative to the body direction of the wearer of the absorbent undergarment 600. The absorbent undergarment 600 can have a waist opening 614 and first and second leg openings 616. In one embodiment, the waist opening 614 can be sewn together and along with fabric layers present therein using a double layered waist band (the layers being the self-fabric or the main body fabric of the absorbent undergarment 600). In some embodiments, the leg openings 616 and the fabric layers joined therein can be sewn using a “self-fabric binding” technique, for example a ⅜ of an inch self-fabric binding at leg opening 616 can be used.

FIGS. 6C and 6D illustrate interior views of the front and back panels of the absorbent undergarment 600, respectively. The absorbent undergarment 600 can have a first absorbent liner 602, in the crotch region and a second absorbent liner 604, extending between the first absorbent liner 602 and the waist opening 614. The second absorbent liner 604 in the back of the absorbent undergarment 600 extends to a slightly larger area than it does in the front of the absorbent undergarment 600. In both front and back panels of the absorbent undergarment 600, the second absorbent liner 604, only partially covers a region between the crotch region and the waist opening 614.

The first absorbent liner 602 can include a plurality of layers. In one embodiment, the layering, structure, materials, and properties of the first absorbent liner 602 is similar to the layering, structure and materials of the absorbent liner 202, as described above in relation to the absorbent undergarment 200. For example, the first absorbent liner 602 can include a top layer made of a cotton and spandex blend with high percentage of cotton (e.g., 95% cotton, 5% spandex), a first and second IHLs, a BWL, for example, a PUL layer, and a bottom layer comprising the self-fabric, which can also be a cotton, spandex blend of high percentage cotton. As with other embodiments, the layers in different regions of the absorbent undergarment 600 that are constructed of the same material can be made of one continuous fabric cut to shape and extending to relevant regions of the absorbent undergarment 600. In other embodiments, a mix of same or different cuts of the fabrics of the same type in various regions can be used.

The second absorbent liner 604 can include a plurality of layers, but may include fewer layers than the first absorbent liner 602 as moisture exposure in the area of the second absorbent liner 604 can be less than the moisture exposure in the area of the first absorbent liner 602 in the crotch region. The second absorbent liner 604 can include a top layer made of a cotton and spandex blend, having a high percentage of cotton (e.g., 95% cotton, 5% spandex), a single IHL, BWL and a bottom layer, comprising the self-fabric. The inclusion of the top layer of the same material as the main body fabric (e.g., a high-cotton-percentage cotton and spandex blend) in the second absorbent liner 604 is intended to mask the existence of the layers below it and make the absorbent undergarment 600 appear unitary and as having been constructed of the same material throughout.

The same technique can also be applied to other embodiments, where the absorbent undergarment includes one or more top layers made of the same material and in the same color and pattern as the main body fabric or the self-fabric, which may also be used as the bottom layer, enveloping the internal layers of the undergarment, in order to make the appearance of the absorbent undergarment a unified and one-piece-constructed undergarment. In other words, the described layers in various regions can be sandwiched between a top layer and a bottom layer of the same fabric, having the same color, and the same pattern to mask the layering inside and between the top and bottom layers. In this context, the top layer and the bottom layer can refer to the inside and outside of the undergarment, respectively, where inside refers to the surface area of the undergarment in contact with the wearer body and the outside refers to the outside of the undergarment, which can be exposed to air, if worn without outer garments, such as pants, shorts and skirts, or the outside can be in contact with outer garments, such as pants, shorts and skirts, worn by the person using the described undergarments. Yet in other embodiments, the unified garment look can be achieved with other techniques than enveloping the garment in the same fabric. For example, some fabrics may be different material, but they may look the same, or can be dyed to look the same. In other examples, various regions of the undergarment can have the same color and pattern in a manner that masks the appearance of different fabric types.

The materials and properties of the IHL and BWL in the absorbent undergarment 600 can be chosen like the material and properties described above in relation to the absorbent undergarments 100-500. In one embodiment, the top layer and the bottom layer are both made of self-fabric material (e.g., a high percentage cotton and spandex blend). In some embodiments, the bottom layer, self-fabric is the same layer and the same fabric as the main body fabric, upon which the other layers are sewn or otherwise assembled.

As illustrated in FIGS. 6C and 6D, the first absorbent liner 602 is in the crotch region of the absorbent undergarment 600. In the front panel of the absorbent undergarment 600, shown in FIG. 6C, the second absorbent liner 604 extends between the crotch region and the waist opening 614, but it only partially covers the region between the first absorbent liner 602 and the waist opening 604. The remaining areas of the absorbent undergarment 600, not covered by the first and second absorbent liners 602, 604, are single layered regions 618 and can be made of the main body fabric.

In some embodiments, the single layered regions 618 and the bottom layers of the first and second absorbent liners 602, 604 are the same layer and the same fabric. Likewise, the first and second absorbent liners 602 and 604 can also share a layer of IHL, a single layer of BWL and the same top layer, while the first absorbent liner 602 can have an extra layer of IHL. In other words, the layers that are common between various regions of the disclosed absorbent undergarments can be cut to extend to and between their common regions and sewn or otherwise attached as such. If a region has additional layers, those layers can be added on top or below the commonly shared layers and fabrics. Yet, in other embodiments, even common fabrics and layers, shared among different regions of the undergarment, can be from separate pieces and sewn together, where appropriate.

The second absorbent liner 604 in the back panel of the absorbent undergarment 600, as shown in FIG. 6D, also extends from the first absorbent liner 602 to the waist opening 614. However, the second absorbent liner 404 extends to a slightly larger region in the back panel of the absorbent undergarment 600, compared to the area it extends to in the front panel of the absorbent undergarment 600. In other words, the second absorbent liner 604 in the front and back panels extends to and between the crotch portion, parts of the leg openings and parts of the waist opening in central regions of the absorbent undergarment 600. Furthermore, the second absorbent liner 604 does not extend to all edges of the waist and the leg openings 614, 616 in front or back panels and the remaining areas uncovered by first and second absorbent liners 602, 604 are single layered regions 618 made of the main body fabric. In one embodiment, the first and second absorbent liners 502 and 504 together cover approximately a 50% perimeter of the leg openings 616, while the second absorbent liner covers approximately a ⅓ perimeter of the waist opening 614.

Men's Boxer

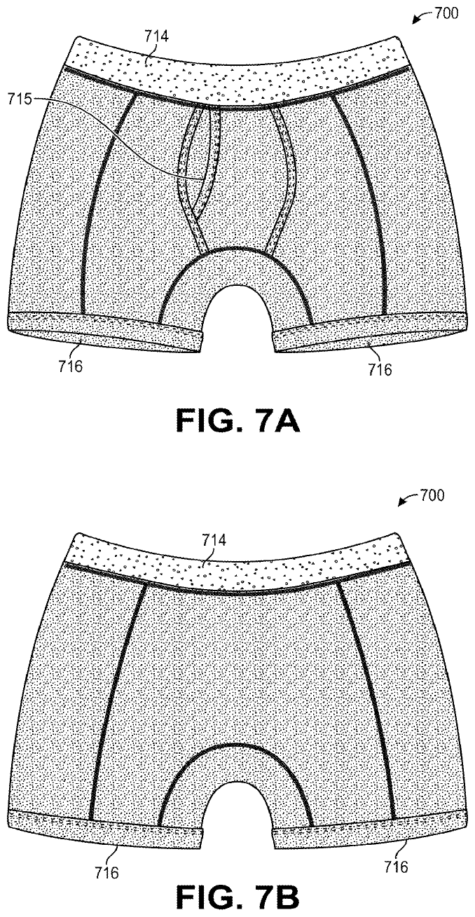

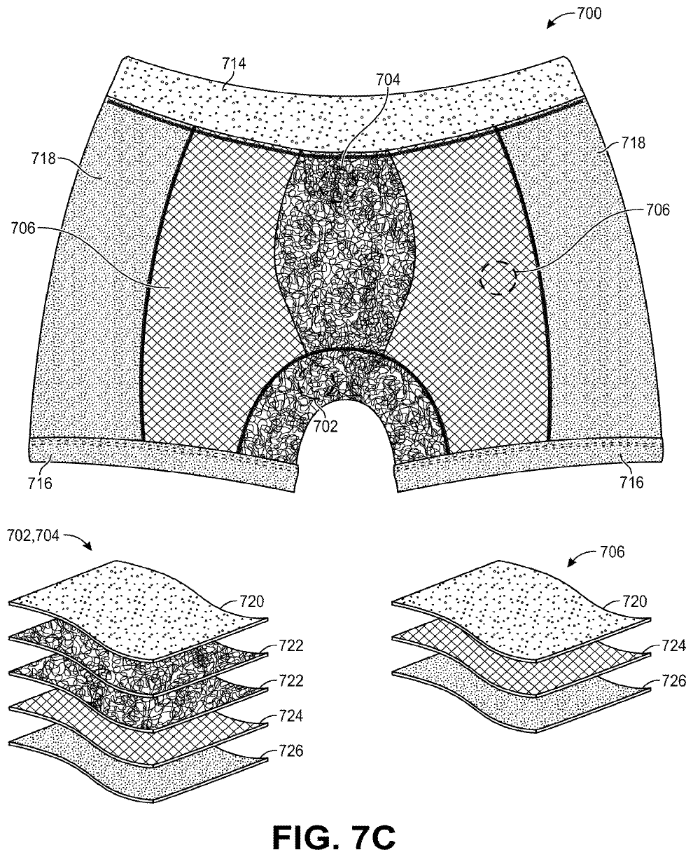

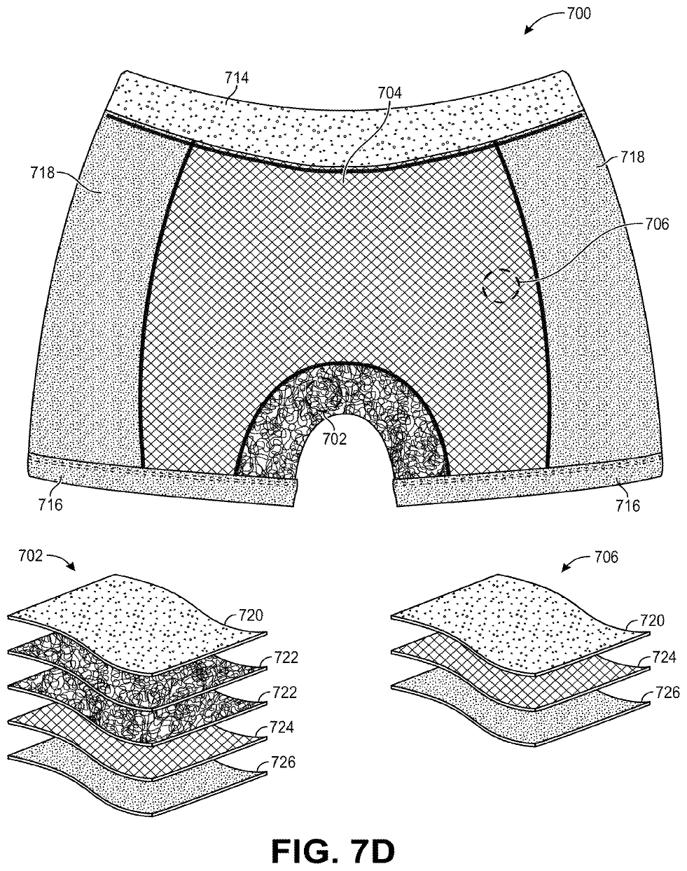

FIGS. 7A-7D show an absorbent undergarment 700 according to an embodiment. The absorbent undergarment 700 can be used by men for incontinence but can also be used by other genders. As will be describe, polyester interacts better with urine, compared to cotton. Consequently, polyester is used in the absorbent undergarment 700 for absorption and in the described layering. The bottom layer is made of self-fabric, upon which other layers can be constructed. In one embodiment, the self-fabric can be a high percentage nylon, nylon and spandex blend (e.g., a brushed 90% nylon, 10% spandex). High-percentage nylon in a blend can refer to at least 80% nylon in the blend.

FIG. 7A illustrates an exterior view of the front of the absorbent undergarment 700. FIG. 7B illustrates an exterior view of the back of the absorbent undergarment 700. Front and back in this context are used relative to the body direction of the wearer of the absorbent undergarment 700. The absorbent undergarment 700 can have a waist opening 714 and first and second leg openings 716. The absorbent undergarment 700 can include an aesthetic-only or functional front panel opening 715. The waist opening 714 can include an elastic (e.g., a brushed elastic) sewn together and along with fabrics that meet at the waist opening 714. In some embodiments, a 5-centimeter brushed elastic at the waist opening 714 can be used. Fold-over elastics can be sewn in the leg openings 716 together and along with fabric layers that meet therein. In one embodiment, a 1.5-centimeter fold-over elastic can be used in the leg openings 716.

FIGS. 7C and 7D illustrate interior views of the front and back panels of the absorbent undergarment 700, respectively. The absorbent undergarment 700 includes a quadruple layered gusset portion 702 in the crotch region of the absorbent undergarment 700. In the front panel of the absorbent undergarment 700, the undergarment includes a quadruple layered liner 704. The quadruple layered liner 704 extends to and between the gusset portion 702 and the waist opening 714 and is flanked in either side by a double layered liner 706. The double layered liner 706 extends to and between the gusset portion 702, the leg openings 716, the quadruple layered liner 704 and the waist opening 714. In some embodiments, the double layered liner 706 does not extend to and does not cover all edges of the waist and leg openings 714, 716. The remaining edges and areas, not covered by the gusset portion 702, quadruple layered liner 704 and double layered liner 706 are single layered regions 718 and can be made of the main body fabric.

The quadruple layered gusset portion and liner 702, 704 can include a plurality of layers. For example, in some embodiments, they can include a top layer 720 made of 100% polyester. Polyester reacts better with urine in terms of absorption, transfer, and odor resistant, compared to other fabrics, and therefore is a preferred fabric contacting the wearers body in regions exposed to urine in an incontinence undergarment. The gusset portion and liner 702, 704 can further include first and second IHLs 722, a BWL 724 and a bottom layer 726. The IHL 722 and BWL 724 can be similar in materials and properties to IHLs and BWLs described above in relation to the absorbent undergarments 100-600. The term quadruple is chosen based on the four layers present above the bottom layer 726. The bottom layer 726 can be made of self-fabric or the main body fabric of the absorbent undergarment 700. Similarly, the double layered liner 706 can also include two layers arranged on top of the bottom layer 726. The double layer liner 706 can include the top layer 720 and the BWL 724. As described earlier, the layers shared between the quadruple layered gusset portion and liner 702, 704, can be from the same piece of fabric. For example, the top layer 720 can be the same piece of fabric extending to both gusset and liner 702, 704. Similarly, the BWL 724 can be a single piece of fabric extending to both gusset 702 and liner 704. Similarly, the bottom layer 726, or self-fabric can be a single piece of fabric extending to gusset 702, liner 704 and other regions of the absorbent undergarment 700. Yet, in other embodiments, these regions can be constructed form different pieces of fabric that are sewn together, even if they may be of the same type of material.

FIG. 7D illustrates an interior back panel view of the absorbent undergarment 700. The back panel of the absorbent undergarment 700 is identical to its front panel, except it does not include a quadruple layered liner, instead the double layered liner 706 extends to and between the quadruple gusset portion 702, and waist and the leg openings 714, 716.

Adaptive Bikini

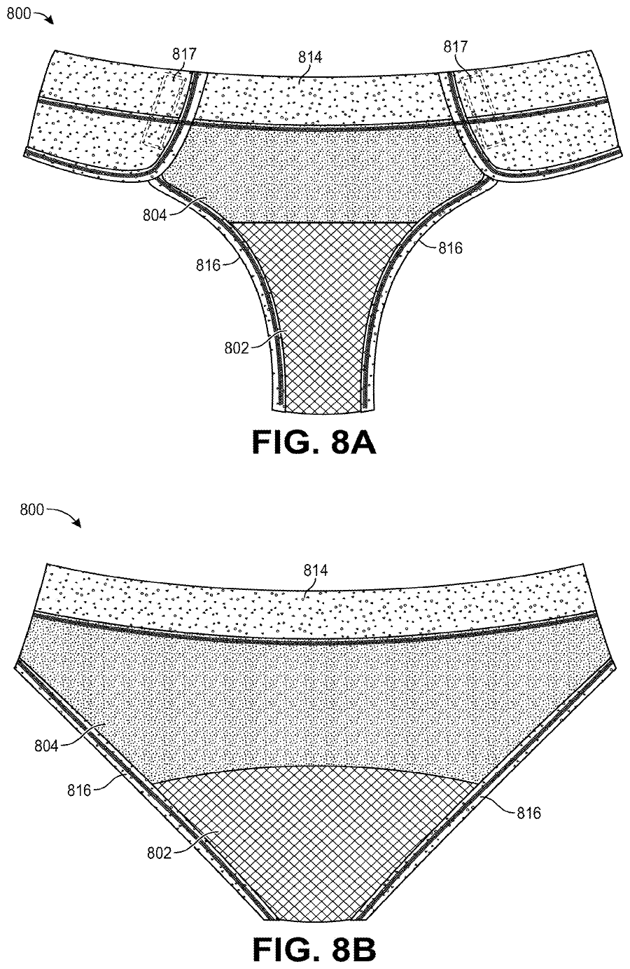

FIGS. 8A-8B illustrate an absorbent undergarment 800 according to an embodiment, termed adaptive bikini. The absorbent undergarment 800 is adaptive because the body of the undergarment comprises of first and second sections, wherein the first section is attachable and detachable to and from the second section, thereby allowing easy access to body parts for cleaning or using the bathroom. Other styles of undergarment, for example those described herein, can also be constructed in the adaptive style of the absorbent undergarment 800. Adaptive style of undergarment can be useful to a large portion of the population, for example, people with disability, mobility issues, or anyone who might have difficulty removing their undergarment for using the bathroom. The attachment of the first and second sections can be implemented by any fastening mechanism 817, such as by Velcro, or other fastening techniques as known by persons of ordinary skill in the art. For example, in one embodiment, ½ by 2 inches hook and loop closure can be used. Furthermore, the edges of the first and/or the second attachable and detachable sections can be lined with fold-over elastic (e.g., a ⅜ of an inch fold-over elastic).

The absorbent undergarment 800 can have an absorbent liner 802 in the crotch region. The absorbent liner 802 can be similar in material and structure to the absorbent liner 102, as described above, in relation to the absorbent undergarment 100. The absorbent undergarment 800 can include an interior front panel, shown in FIG. 8A and an interior back panel, shown in FIG. 8B. The absorbent undergarment 800 can include a lined portion 804, which can be constructed of an additional layer of the main body fabric from which the absorbent undergarment 800 is constructed. In the front panel of the absorbent undergarment 800, shown in FIG. 8A, the lined portion 804 reaches some portions of the waist opening 814 and some portions of the leg openings 816.

In the interior back panel shown in FIG. 8B, the lined portion 804 extends to all edges of the waist opening 814 and to all edges of the leg openings 816, not reached or covered by the crotch region or the absorbent liner 802. In other words, in the back panel, the crotch region, or the absorbent liner 802 reaches and covers a portion of the leg openings 816 and the remaining edges of the leg openings 816 are covered by the lined portion 804. In some embodiments, the crotch region, or the absorbent liner 802 covers ½ perimeter of the leg openings 816 and the other halves are covered by the lined portion 804. Elastics and fold-over elastics sewn in the seams can also be used in the waist and leg openings 814, 816. DTM technique and measurements, as disclosed above in relation to the absorbent undergarment 100 can also be used.

The absorbent undergarment 800 can be made of a main body fabric that is appropriate for use in water, for example, a quick drying fabric. In a preferred embodiment, the main body fabric or self-fabric of the absorbent undergarment 800 can be a nylon and spandex blend having a high percentage (e.g., at least 80%) nylon. An example includes a brushed microfabric, self-fabric, having 90% nylon and 10% spandex.

The absorbent liner 802 can be similar in layers and materials therein to the absorbent liner 102, as described above in relation to the absorbent undergarment 100. For example, the absorbent layer 802 can include a top layer made of cotton and spandex blend having a high percentage of cotton (e.g., at least 80%), first and second IHLs, a BWL (e.g., a PUL), and a bottom layer made of the self-fabric or the main body fabric.

The first attachable and detachable section comprises generally the side regions of the absorbent undergarment 800, while the second attachable and detachable body section can comprise generally the crotch and central regions of the absorbent undergarment 800, such that detaching the first and second sections from one another can allow for easy access to body parts (e.g., genitalia). In some embodiments, the first section folds over the second section when attached, and in other embodiments, the second section folds over the first section when attached. The fastening mechanism 817 can exist in one attachment point between the first and second sections, or it can exist in a plurality of locations between the first and second sections. A preferred embodiment includes two fastening mechanisms 817 between the first and second sections on the left and right side of the absorbent undergarment 800. In some embodiments, the first or the second section can include one or more pull-tabs (e.g., an elastic loop pull tab), which can further assist the wearer in detaching the first and second sections from one another. The pull-tab can be DTMed to match the general color and pattern of the absorbent undergarment 800. In some embodiments, the waistband of the absorbent undergarment 800 can include a double layered waistband. As an example, a ⅜ of an inch elastic with ⅛ of an inch double needle cover stitch in the waist opening 814 can be used to sew the fabric layers that meet therein.

Thong

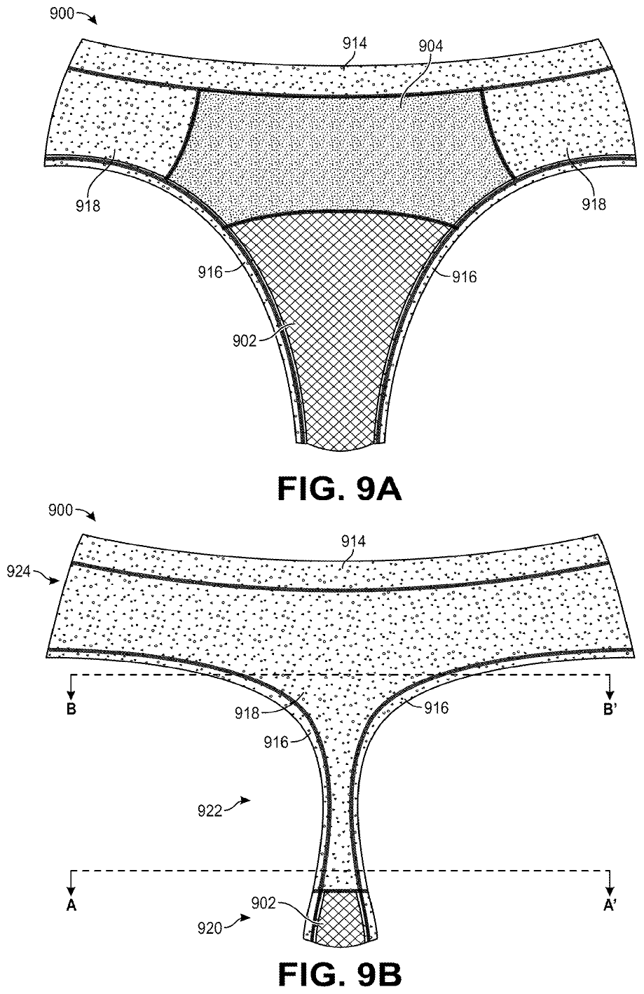

FIGS. 9A-9B illustrate an absorbent undergarment 900 according to an embodiment, in a garment style termed, thong, which can be used by women, but other gender groups can also use this undergarment style. FIGS. 9A, 9B illustrate interior front and back panels of the absorbent undergarment 900, respectively. The absorbent undergarment 900 can have an absorbent liner 902 in the crotch region. The absorbent liner 902 can be similar in material and structure to the absorbent liner 102, as described above in relation to the absorbent undergarment 100. The absorbent undergarment 900 can include a lined portion 904, which can be constructed of an additional layer of the main body fabric from which the absorbent undergarment 900 is constructed. In the front panel, the absorbent undergarment 900 can include single layered regions 918 flanking the lined portion 904. In the front panel of the absorbent undergarment 900, shown in FIG. 9A, the lined portion 904 reaches some portions of the waist opening 914 and some portions of the leg openings 916. The remaining portions are covered by the single layered region 918.

The interior back panel of the absorbent undergarment 900 comprises of a substantially narrower surface area compared to the front panel of the absorbent undergarment 900. For example, the back panel of the absorbent undergarment 900 can be approximately at least 50% less surface area than the front panel. In the interior back panel shown in FIG. 9B, the absorbent liner 902 reaches a few inches above the crotch region (e.g., approximately 2 inches). In other words, the absorbent liner 902 in the front panel continues to wrap around to the back panel and rising the back panel by a few inches. In back panel, the absorbent undergarment 900 can be visually divided into three regions: region 920 below line A-A′, region 922 above line A-A′ and below line B-B′ and region 924 above the line B-B′. In one embodiment, the width of the absorbent undergarment 900 in the region 920 is slightly larger than the width of the absorbent undergarment 900 in the region 922 (for example by a factor of 2) to allow for a more robust absorbent liner 902. The areas of the back panel of the absorbent undergarment 900, not covered by the absorbent liner 902 are single layered regions 918 made of the main body fabric or self-fabric. The self-fabric can be a high cotton percentage cotton, spandex blend (e.g., at least 80% cotton).

Elastics and fold-over elastics sewn in the seams can also be used in the waist and leg openings 914, 916. DTM techniques and measurements, as disclosed herein can also be used in absorbent undergarment 900. For example, the waist opening 914 can be sewn with a one-inch elastic covered with self-fabric. The leg openings can be sewn with ⅜ of an inch fold-over elastic DTM, together and long with fabric layers meeting therein.

Alternative Sleeper Boyshort

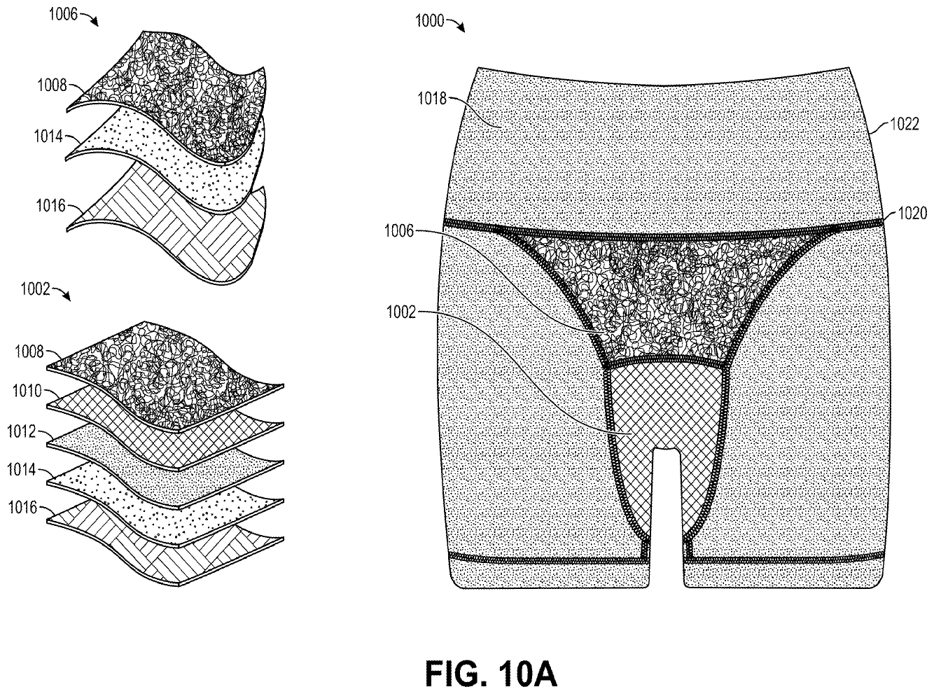

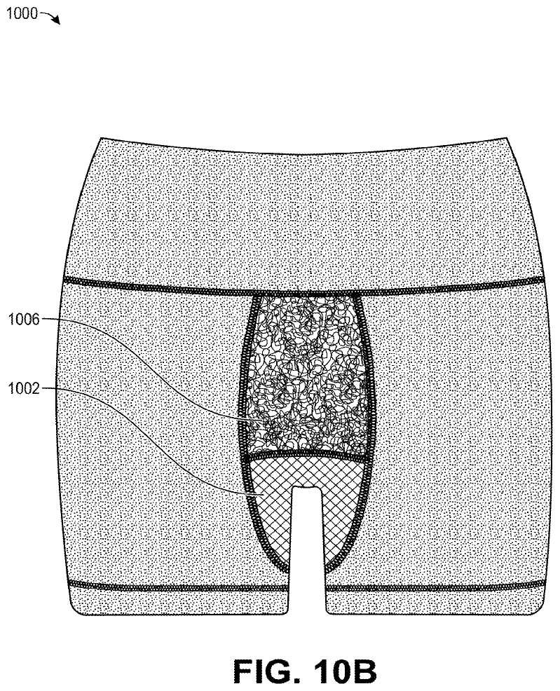

FIGS. 10A-10B illustrate an absorbent undergarment 1000 according to an embodiment, also in the garment style termed, sleeper boyshort, which can be used by women, but other gender groups can also use this undergarment style. FIGS. 10A and 10B illustrate interior views of the front and back panels of the undergarment 1000, respectively. The undergarment 1000 can include an outer shell or main body made of a cotton/spandex blend, having a cotton percentage of at least 80%. In some embodiments, a 95% cotton and 5% spandex can be used for the self-fabric. The undergarment 1000 can include an absorbent liner in the crotch portion. In one embodiment, the absorbent liner in the crotch portion includes a gusset portion 1002. The gusset portion 1002 can be constructed of a variety of layers of fabric according to the embodiments described herein and sewn or otherwise be adhered to the self-fabric, main body fabric or a shell in the crotch portion. In that respect, the self-fabric or the main body fabric can become a bottom layer upon which a gusset portion 1002 can be constructed by sewing, or other bonding mechanisms. The gusset portion 1002 can include the layering, as shown in FIG. 10A. The gusset portion 1002 can include the same layering in the back panel. The back panel of the undergarment 1000 is shown in FIG. 10B. The example layering, as shown in FIG. 10A, includes the layers in the gusset portion 1002 from the most interior layer 1008 closes to the skin of a wearer of the undergarment 1000 to the main body fabric, the most exterior layer 1016. Example layering in the gusset portion 1002 can include top layer 1008, made of a high percentage cotton and spandex blend (for example, above 80% cotton). In one embodiment, a blend of 95% cotton and 5% spandex is used. The top layer 1008 is the layer closest to a wearer's body. Next, two IHLs 1010 and 1012 can be used. The construction and material of the IHLs 1010 and 1012 are similar as those described in relation to the above embodiments. For example, the IHLs 1010 and 1012 can include a towel-like fabric made of a polyester material, wherein the towel-like fabric has a textured surface area, having a larger surface area than other layers, including the top layer 1008. Next layer further away from the body of the wearer, includes a BWL 1014, made of similar material as described above in relation to the other embodiments. For example, the BWL 1014 can be a knit polyester fabric layer with a polyurethane laminate coating. Next layer furthest away from the body of the wearer of the undergarment 1000 is the main body fabric, or self-fabric, or the bottom layer, made of a high percentage cotton and spandex blend in some embodiments (e.g., higher than 80% cotton, or 95% cotton in some embodiments). A waistband 1018 can be constructed from folding over the self-fabric on itself, making a circular shape from the fold-over and then sewing the waistband 1018 at seams 1020 and 1022.

The undergarment 1000 can also include a lined portion 1006. In some embodiments, the lined portion 1006 can include a layering of a top layer 1008, and the main body fabric layer 1016, enveloping the BWL 1014. In the sleeper boyshort style, the lined portion extends from the gusset portion to the waist opening. To provide more leakage protection in the front panel of the undergarment 1000, where the chances of a leak occurring is higher, the lined portion 1006 covers a larger surface area in the front panel of the undergarment 1000 than it does in the back panel of the undergarment 1000. As can be appreciated from the above description, the lined portion and the gusset portion share the top layer 1008, the BWL 1014 and the main body fabric 1016. This can be accomplished at the cutting stage, where the selected patterns are cut and sewn in a manner that the shared layers are constructed as one piece and the additional layers of the gusset portion 1002 are inserted therein and the piece is sewn or otherwise bonded in the crotch portion of the undergarment 1000. In other embodiments, the gusset portion 1002 and the lined portion 1006 can be constructed separately and sewn or otherwise attached to the crotch portion of the undergarment 1000 in the crotch portion. In each scenario, the main body fabric that is indicated as the layer 1016 can be prefabricated as a shell or the main body of the undergarment 1000 upon which the gusset portion 1002 and the lined portion 1006 are attached in the appropriate regions.

Alternative Boxer

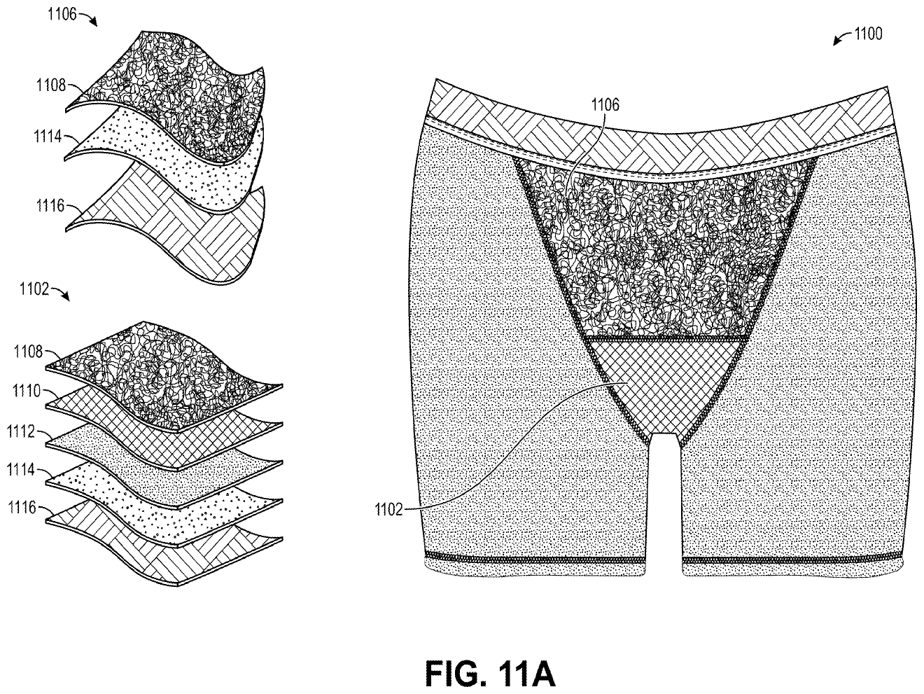

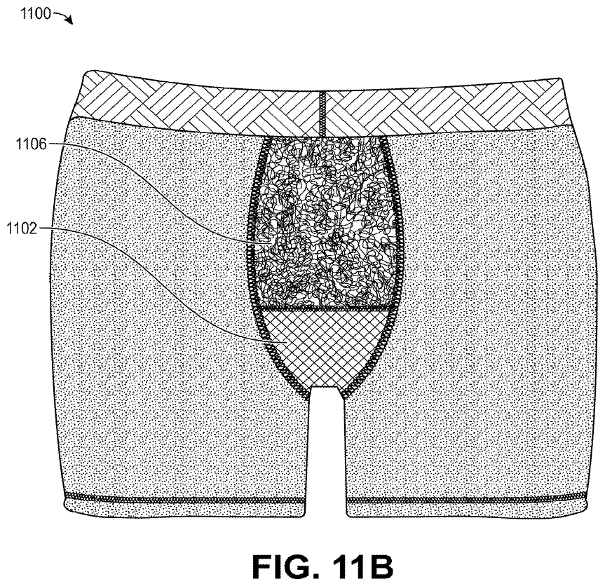

FIGS. 11A-11B illustrate an absorbent undergarment 1100 in a garment style also termed, boxer, which can be used by transgender women, but other gender groups can also use this undergarment style. The undergarment 1100 is similar in construction to the undergarment 1000, described above in relation to the embodiment of FIG. 10. FIGS. 11A and 11B, respectively, shown an interior view of a front and back panel of the undergarment 1100. The undergarment 1100, can include a gusset portion 1102 and a lined portion 1106, similar in construction and material to those described in relation to the gusset portion 1002 and the lined portion 1006 in the embodiment of FIG. 10. Similar to the embodiment of FIG. 10, the lined portion 1106 can cover a larger surface area in the front panel than it does in the back panel to provide additional leakage protection in the front area of the undergarment, which can be more likely to be exposed to bodily fluids.

Improved Method of Manufacturing

The described garments can be produced more efficiently by employing a computerized manufacturing process, such as computerized circular knitting. A computerized circular knitting machine (CCK machine) can receive an input indicating the pattern, textures and styles selected for a shell, main body and/or exterior of a garment. In one embodiment, the CCK machine receives that input and produces the garment in the selected style and texture. For example, the main body portion and/or the shell portion of a described garment can be produced by the CCK machine, according to inputs given to the CCK machine regarding the patterns and textures of the various regions of the main body and/or the shell. The crotch portion, lined portions and/or other non-shell, non-main body portions, such as the layered portions of the garment can then be applied manually by sewing or other bonding techniques to finish the garment according to the described embodiments. For example, in some embodiments, the CCK machine can produce the main body of an undergarment having various textures and patterns in different regions of the main body. The crotch portion is then applied manually and sewn onto a front panel and a back panel of the undergarment. The leg bindings in the crotch portion can also be manually applied by sewing or other bonding technique. In some embodiments, elastics at the leg openings and/or the waist opening can also be used to finish the garment. A computerized manufacturing process, such as those done by CCK machines can be used with any of the styles and garments, described herein.

An advantage of utilizing a computerized manufacturing process, including a CCK machine, is that a produced garment can be partially or fully seamless, where although various regions of the garment might have different textures and patterns, the garment can be partially or substantially seamless. Furthermore, since the CCK machine can produce the entire main body as one piece, some seams can be eliminated compared to traditional techniques of cut and sew. For example, an underwear produced using traditional cut and sew might have side seams on the hip portions and two crotch seams in the crotch region. These and other main body seams can be reduced or eliminated by using a CCK machine in combination with the described embodiments. Another advantage is that the garment produced using a computerized knitting technique can be more stretchable and comfortable than garments produced using other methods because of the use of the knitted textures. The CCK machine can also reduce the manual labor that would otherwise be required for manufacturing garments.

While the invention has been particularly shown and described with reference to specific embodiments thereof, it should be understood that changes in the form and details of the disclosed embodiments may be made without departing from the scope of the invention. Although various advantages, aspects, and objects of the present invention have been discussed herein with reference to various embodiments, it will be understood that the scope of the invention should not be limited by reference to such advantages, aspects, and objects. Rather, the scope of the invention should be determined with reference to patent claims.

What is claimed is:

1. An undergarment comprising:
a waist opening;
first and second leg openings;
a main body portion made of a main body fabric, the main body portion comprising a crotch portion, wherein the crotch portion comprises portions of the first and second leg openings;
the crotch portion further comprising an absorbent liner, the absorbent liner comprising:
a top layer;
one or more intermediate hydrophilic layers;
a breathable and waterproof layer;
a self-fabric layer made of the main body fabric.

2. The undergarment of claim 1, wherein
the main body fabric comprises a cotton and spandex blend with at least 80% cotton;
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating; and
wherein the undergarment comprises a front panel and a back panel, the main body portion in a portion extending between the waist opening, the leg openings and the crotch portion is lined with an additional layer of the main body fabric, wherein the lined portions exist in both front and back panels only partially and not extending to all edges of the waist and leg openings.

3. The undergarment of claim 1, wherein
the main body fabric comprises a cotton and spandex blend with at least 80% cotton;
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating; and
wherein the undergarment comprises a front panel and a back panel, the main body portion in a portion extending between the waist opening, the leg openings and the crotch portion is lined with an additional layer of the main body fabric, wherein the lined portion in the front panel only extends to portions of the edges of the waist and leg openings and the lined portion in the back panel extends to edges of the waist opening in the back panel and edges of the leg openings in the back panel not reached by the crotch portion.

4. The undergarment of claim 1, wherein
the main body fabric comprises a cotton and spandex blend with at least 80% cotton, the top layer and the self-fabric layer are made of the main body fabric;
the absorbent liner comprises a gusset portion and a lined portion,
the gusset portion comprises two intermediate hydrophilic layers;
the lined portion comprises a single hydrophilic layer;
wherein the intermediate hydrophilic layers in the gusset and the lined portions comprise a towel-like fabric made of a polyester material, wherein the towel-like fabric has a textured surface, having a larger surface area than the top layer and the breathable and waterproof layer comprises a knit polyester fabric layer with a polyurethane laminate coating; and
the lined portion extends from the gusset portion to the waist and leg openings in partial edges of the waist and the leg openings and not extending to all edges of the waist and leg openings.

5. The undergarment of claim 1, wherein
the main body fabric comprises a cotton and spandex blend with at least 80% cotton;
wherein the absorbent liner is a first absorbent liner;
in the first absorbent liner,
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating; and
a second absorbent liner extending between the first absorbent liner and the waist opening, the second absorbent liner comprising:
a second absorbent layer top layer, comprising a cotton and spandex blend with at least 80% cotton;
a second absorbent layer intermediate hydrophilic layer, comprising a towel-like fabric made of a polyester material, wherein the towel-like fabric has a textured surface, having a larger surface area than the surface area of the second absorbent layer top layer; and
the self-fabric;
wherein the undergarment comprises a front panel and a back panel, and the second absorbent liner in the front and back panels extends between the crotch portion and the waist opening, wherein the extension area in the back panel and through the waist opening edges are larger than the extension in the front panel.

6. The undergarment of claim 1, wherein
the main body fabric comprises a cotton and spandex blend with at least 80% cotton;
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating;
wherein the undergarment comprises a front panel and a back panel, the main body portion in the back panel is lined with an additional layer of the main body fabric, wherein the lined portion extends to the waist opening in the back panel and edges of the leg openings in the back panel not shared with the crotch portion.

7. The undergarment of claim 1, wherein
the main body fabric comprises a cotton and spandex blend with at least 80% cotton;
wherein the absorbent liner is a first absorbent liner;
in the first absorbent liner,
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating; and
a second absorbent liner extending between the first absorbent liner and the waist opening, the second absorbent liner comprising:
a second absorbent layer top layer, comprising a towel-like fabric made of a polyester material, wherein the towel-like fabric has a textured surface, having a larger surface area than the surface area of the main body fabric; and
the self-fabric;
wherein the undergarment comprises a front panel and a back panel, and the second absorbent liner in the front and back panels extends to and between the crotch portion, parts of the leg openings and parts of the waist opening in central regions of the undergarment, and the second absorbent liner does not extend to all edges of the waist and the leg openings in front or back panels.

8. The undergarment of claim 1, wherein
the undergarment comprises a front panel and a back panel;
the main body fabric comprises a nylon and spandex blend having at least 80% nylon;
the absorbent liner comprises a quadruple layered gusset portion in the crutch portion of the undergarment, a quadruple layered liner in the front panel of the undergarment, extending to and between the gusset portion and the waist opening and flanked by a double layered liner, wherein the double layered liner extends to and between the gusset portion, the leg opening, the quadruple layered liner and the waist opening, the double layered liner in both front and back panels not extending to all edges of the waist and leg openings;
wherein the quadruple layered gusset portion and the quadruple layered liner, comprise:
the top layer, first and second intermediate hydrophilic layers, and the breathable waterproof layer;
the double layered portion comprises the top layer and the breathable waterproof layer,
wherein the top layer comprises a polyester fabric, the intermediate hydrophilic layers each comprise a towel-like fabric made of a polyester material, wherein the towel-like fabric has a textured surface, having a larger surface area than the top layer and the breathable and waterproof layer comprises a knit polyester fabric layer with a polyurethane laminate coating.

9. The undergarment of claim 1, wherein
the main body fabric comprises a nylon and spandex blend having at least 80% nylon;
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating; and
wherein the undergarment comprises a front panel and a back panel, the main body portion in a portion extending between the waist opening, the leg openings and the crotch portion is lined with an additional layer of the main body fabric, wherein the lined portion in the front panel only extends to some portions of the edges of the waist and leg openings and the lined portion in the back panel extends to all edges of the waist opening in the back panel and edges of the leg openings in the back panel not shared with the crotch portion,
wherein the body portion comprises first and second sections, wherein the first section is attachable and detachable to and from the second section.

10. The undergarment of claim 1, wherein
the main body fabric comprises a cotton and spandex blend with at least 80% cotton;
the top layer comprises a cotton and spandex blend with at least 80% cotton;
the intermediate hydrophilic layers comprise a first and second towel-like fabric made of a polyester material, wherein the towel-like fabric has a textured surface, having a larger surface area than the top layer;
the breathable and waterproof layer comprises a knit polyester fabric layer with a polyurethane laminate coating; and
wherein the undergarment comprises a front panel and a narrow back panel, the main body portion in a portion extending between the waist opening, the leg openings and the crotch portion is lined with an additional layer of the main body fabric, wherein the lined portions exist in front panel only and extending to some portions of edges of the waist and leg openings.

11. An absorbent undergarment, comprising:
a waist opening;
first and second leg openings;
a main body fabric extending to all regions of the absorbent undergarment and comprising a bottom layer of the absorbent undergarment, wherein the bottom layer comprises the outside surfaces of the absorbent undergarment; and
an absorbent liner in a crotch region of the absorbent undergarment, the absorbent liner comprising:
a top layer;
one or more intermediate hydrophilic layers;
a breathable, waterproof layer; and
the bottom layer,
wherein the top and bottom layers envelope the one or more hydrophilic layers and the breathable, waterproof layer.

12. The absorbent undergarment of claim 11, wherein
the top and bottom layers comprise a cotton and spandex blend of at least 80% cotton;
the one or more hydrophilic layers comprise first and second towel-textured layers each made of a polyester fabric having a double-sided velour fabric; and
the breathable, waterproof layer comprises a knit polyester fabric with a polyurethane laminate coating.

13. The absorbent undergarment of claim 11 further comprising regions in front and back of the absorbent undergarment lined with an additional layer of the main body fabric.

14. The absorbent undergarment of claim 11, wherein the main body fabric comprises a cotton and spandex blend, having at least 80% cotton.

15. The absorbent undergarment of claim 11, wherein
the breathable, waterproof layer comprises a knit polyester fabric with a polyurethane laminate coating;
the hydrophilic layers, each comprise a towel-textured surface area on either side, and each hydrophilic layer is made of a polyester velour fabric;
the top layer comprises one of the hydrophilic layers;
the absorbent liner further comprises a gusset portion, comprising the hydrophilic layers; and
the absorbent undergarment further comprises a lined portion extending from the gusset portion in the crotch region to the waist opening and the leg openings, wherein the top layer and the breathable waterproof layer extend over the lined portion.

16. The absorbent undergarment of claim 11, wherein
the bottom layer is made of a cotton, spandex blend having at least 80% cotton;
the breathable, waterproof layer comprises a knit polyester fabric with a polyurethane laminate coating;
the hydrophilic layers, each comprise a towel-textured surface area on either side, and each hydrophilic layer is made of a polyester velour fabric;
the absorbent liner comprises a first absorbent liner, wherein the first absorbent liner comprises:
the top layer made of a cotton, spandex blend having at least 80% cotton;
first and second hydrophilic layers;
the breathable, waterproof layer; and
the absorbent undergarment further comprises a second absorbent liner, extending from the first absorbent liner from the crotch region to the waist opening, wherein the second absorbent liner comprises:
the top layer;
the first and second hydrophilic layers; and
the breathable, waterproof layer.

17. The absorbent undergarment of claim 11, wherein an elastic is sewn in the leg openings using fold-over technique.

18. The absorbent undergarment of claim 11, comprising a front panel and a back panel, wherein the front panel is attachable and detachable to and from the back panel.

19. The absorbent undergarment of claim 11, wherein
the bottom layer is made of a nylon, spandex blend having at least 80% nylon;
the breathable, waterproof layer comprises a knit polyester fabric with a polyurethane laminate coating;
the hydrophilic layers, each comprise a towel-textured surface area on either side, and each hydrophilic layer is made of a polyester velour fabric;
the absorbent liner comprises a gusset in the crotch region of the absorbent undergarment, wherein the gusset comprises:
the top layer made of 100% polyester;
first and second hydrophilic layers;
the breathable, waterproof layer; and
the absorbent undergarment further comprises a second absorbent liner, extending in a front panel of the absorbent undergarment, from the gusset in the crotch region to the waist opening, wherein the second absorbent liner comprises:
the top layer made of 100% polyester;
the first and second hydrophilic layers;
the breathable, waterproof layer; and
the bottom layer;
the absorbent undergarment further comprises a third absorbent liner flanking the second absorbent liner in the front panel of the absorbent undergarment and comprising:
the top layer made of 100% polyester;
the breathable, waterproof layer; and
the bottom layer, wherein the third absorbent liner in a back panel of the absorbent undergarment extends from the gusset in the crotch to the waist opening, and leg openings, covering a central region of the absorbent undergarment in the back panel.

20. The absorbent undergarment of claim 11, wherein
the bottom layer is made of a cotton, spandex blend having at least 80% cotton;
the breathable, waterproof layer comprises a knit polyester fabric with a polyurethane laminate coating;
the hydrophilic layers, each comprise a towel-textured surface area on either side, and each hydrophilic layer is made of a polyester velour fabric;
the absorbent liner comprises a first absorbent liner, wherein the first absorbent liner comprises:
the top layer made of a cotton, spandex blend having at least 80% cotton;
first and second hydrophilic layers;
the breathable, waterproof layer; and
the absorbent undergarment further comprises a second absorbent liner, extending in both front and back panels of the absorbent undergarment, from the first absorbent liner and from the crotch region to the waist opening and the leg openings, wherein the second absorbent liner comprises:
one of the hydrophilic layers; and
the breathable, waterproof layer.

21. The undergarment of claim 1,
wherein the main body fabric comprises a cotton and spandex blend with at least 80% cotton, the top layer and the self-fabric layer are made of the main body fabric;
the absorbent liner comprises a gusset portion,
wherein the gusset portion comprises the top layer, two intermediate hydrophilic layers, the breathable and waterproof layer, and the self-fabric;
wherein the intermediate hydrophilic layers comprise a towel-like fabric made of a polyester material, wherein the towel-like fabric has a textured surface, having a larger surface area than the top layer and the breathable and waterproof layer comprises a knit polyester fabric layer with a polyurethane laminate coating; and
wherein the undergarment further comprises a lined portion, comprising the self-fabric, and the top layer, sandwiching the breathable and waterproof layer, wherein the lined portion extends from the gusset portion to the waist opening, covering a larger area in a front panel of the undergarment than a back panel of the undergarment.
